(19) United States
(12) Patent Application Publication
(10) Pub. No.: US 2018/0231506 A1
(43) Pub. Date: Aug. 16, 2018

(54) LIGHT TRANSMITTING APPARATUS, LIGHT TRANSMITTING METHOD, AND OBJECT INFORMATION ACQUIRING APPARATUS

(71) Applicant: CANON KABUSHIKI KAISHA, Tokyo (JP)

(72) Inventor: Shigeru Ichihara, Tokyo (JP)

(21) Appl. No.: 15/892,612

(22) Filed: Feb. 9, 2018

(30) Foreign Application Priority Data
Feb. 16, 2017 (JP) 2017-026677

(51) Int. Cl. G01N 29/24; F21V 8/00; G02B 6/28; G02B 6/42; A61B 5/00

(52) U.S. Cl. CPC G01N 29/2462; G02B 6/0006; A61B 5/0095; G02B 6/4298; G01N 29/2418; G02B 6/2808

(57) ABSTRACT

A light transmitting apparatus, comprises a bundle fiber configured to include a plurality of strand groups; and a control unit which controls an incidence position of light incident on an incident end-side cross-section of the bundle fiber, wherein in the bundle fiber, the plurality of strand groups are arranged so as to respectively form a plurality of incident regions on the incident end-side cross-section, and a plurality of exit regions respectively corresponding to the strand groups are arranged in a different layout from the plurality of incident regions on a cross-section on an exit end side, and the control unit is configured to be capable of changing a region, to which transmitted light is to be incident, among the plurality of incident regions by controlling the incidence position of light.

### BACKGROUND OF THE INVENTION

### Field of the Invention

The present invention relates to a light transmitting apparatus using a bundle fiber.

### Description of the Related Art

Recently, in the field of medicine, research is underway on imaging of functional information of the inside of an object such as structural information and physiological information. Photoacoustic tomography (PAT) is recently being proposed as such an imaging technique.

When a living organism that is an object is irradiated with light such as laser light, an acoustic wave (typically, an ultrasonic wave) is generated as the light is absorbed by living tissue inside the object. This phenomenon is referred to as a photoacoustic effect and an acoustic wave generated by a photoacoustic effect is referred to as a photoacoustic wave. Since tissues constituting an object have respectively different absorption rates of optical energy, sound pressure of generated photoacoustic waves also differ. With PAT, by receiving a generated photoacoustic wave with a probe and mathematically analyzing a received signal, characteristic information inside an object can be acquired.

In an apparatus using photoacoustic tomography, it is known that acquiring a photoacoustic wave signal generated inside an object requires optimization of both a position where light is irradiated and an arrangement position of a probe.

BIOMEDICAL OPTICS EXPRESS, Vol. 5, No. 11 (2014), 3765 discloses a technique in which a bundle fiber is used as a light transmitting member and an exit end of the bundle fiber is moved in a vicinity of a probe to select an optimal irradiation position.

In addition, Japanese Patent Application Laid-open No. 2016-049212 discloses providing a mechanism which changes a light irradiation direction to a vicinity of a probe in order to efficiently guide light to a desired position.

### SUMMARY OF THE INVENTION

In photoacoustic wave measurement, an optimal light irradiation position is dependent on a living organism that is a measurement target. Therefore, an apparatus performing photoacoustic measurement favorably enables a light irradiation position to be selected at will. However, as described in BIOMEDICAL OPTICS EXPRESS, Vol. 5, No. 11 (2014), 3765, adopting a configuration in which the exit end of a bundle fiber is moved requires providing a complex movement mechanism and creates a problem in that a configuration, a control system, and the like in a periphery of a probe become complicated.

The present invention has been made in consideration of such problems existing in prior art and an object thereof is to provide a light transmitting apparatus which enables a light irradiation position to be selected with a simple configuration.

The present invention in its one aspect provides a light transmitting apparatus, comprising a bundle fiber configured to include a plurality of strand groups, each of which is an assembly of fiber strands; and a luminous flux control unit which controls an incidence position of light incident on an incident end-side cross-section of the bundle fiber, wherein in the bundle fiber, the plurality of strand groups are arranged so as to respectively form a plurality of incident regions on the incident end-side cross-section, and a plurality of exit regions respectively corresponding to the strand groups are arranged in a different layout from the plurality of incident regions on a cross-section on an exit end side of light of the bundle fiber, and the luminous flux control unit is configured to be capable of changing a region, to which transmitted light is to be incident, among the plurality of incident regions by controlling the incidence position of light.

The present invention in its another aspect provides a light transmitting apparatus including a bundle fiber configured to include a plurality of strand groups, each of which is an assembly of fiber strands, wherein in the bundle fiber, the plurality of strand groups are arranged so as to respectively form a plurality of incident regions on an incident end-side cross-section, a plurality of exit regions respectively corresponding to the strand groups are arranged in a different layout from the plurality of incident regions on an exit end side, and in accordance with a change of the incident region, the exit region changes without changing a position of the exit end.

The present invention in its another aspect provides a method of transmitting light using a bundle fiber in which a plurality of fiber strand groups are arranged so as to respectively form a plurality of incident regions on an incident end-side cross-section and a plurality of exit regions respectively corresponding to the fiber strand groups are arranged in a different layout from the plurality of incident regions on an exit end side, the method comprising selecting the incident region to which transmitted light is to be incident; and performing light transmission by causing light to be incident to the selected region, wherein in accordance with the selected incident region, the light is emitted from the exit end side in a different light distribution without changing a position of the exit end.

According to the present invention, a light irradiation position can be selected with a simple configuration.

Further features of the present invention will become apparent from the following description of exemplary embodiments with reference to the attached drawings.

### BRIEF DESCRIPTION OF THE DRAWINGS

FIG. 1 is a schematic diagram of a light transmitting apparatus according to a first embodiment of the present invention;

FIG. 2 is a schematic diagram showing an incident end surface of a bundle fiber according to the first embodiment of the present invention;

FIG. 3 is a schematic diagram showing an incident end surface of a bundle fiber according to the first embodiment of the present invention;

FIG. 4 is a schematic diagram showing an incident end surface of a bundle fiber according to the first embodiment of the present invention;

FIG. 5 is a configuration diagram of a photoacoustic apparatus according to a second embodiment of the present invention;

FIG. 6 shows a modification of a photoacoustic apparatus according to the present invention;

FIG. 7 is a diagram explaining a configuration of a bundle fiber according to Example 1 of the present invention;

FIG. 8 is a diagram explaining a configuration of a bundle fiber according to Example 2 of the present invention;

FIG. 9 is a diagram explaining a configuration of a bundle fiber according to Example 3 of the present invention; and

FIG. 10 is a diagram explaining a configuration of a bundle fiber according to Example 4 of the present invention.

### DESCRIPTION OF THE EMBODIMENTS

Hereinafter, preferred embodiments of the present invention will be described with reference to the drawings. However, it is to be understood that dimensions, materials, shapes, relative arrangements, and the like of components described below are intended to be changed as deemed appropriate in accordance with configurations and various conditions of apparatuses to which the present invention is to be applied. Therefore, the scope of the present invention is not intended to be limited to the embodiments described below.

### First Embodiment

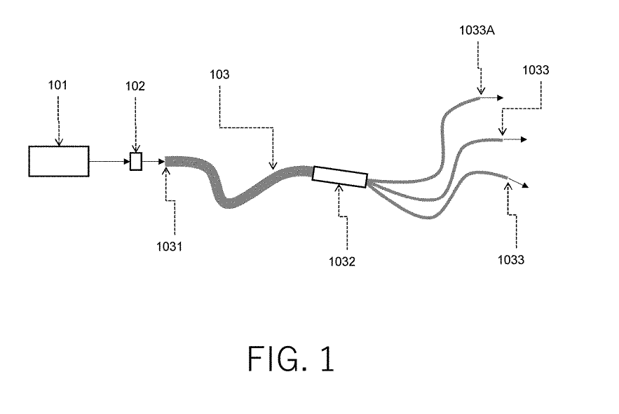

FIG. 1 shows a mode of a light transmitting apparatus according to a first embodiment.

The light transmitting apparatus according to the first embodiment is configured to include a light source unit 101, a luminous flux control unit 102, and a bundle fiber 103. In addition, the bundle fiber 103 includes an incident end 1031, a mixing branch portion 1032, and a plurality of exit ends 1033. Moreover, while FIG. 1 shows one incident end 1031 and three exit ends 1033, the numbers of the incident end and the exit end are not limited thereto.

The light source unit 101 is an apparatus that generates pulse light for irradiating an object. While the light source is desirably a laser light source in order to obtain a large output, a light-emitting diode, a flash lamp, or the like can be used in place of a laser. When using a laser as the light source, various lasers such as a solid-state laser, a gas laser, a dye laser, and a semiconductor laser can be used.

In addition, desirably, a wavelength of the pulse light is a specific wavelength which is absorbed by a specific component among components constituting the object and which enables light to propagate to the inside of the object. Specifically, when the object is a living organism, light with a wavelength of at least 600 nm and not more than 1500 nm is desirably used. Since light in this range is capable of reaching relatively deep portions of a living organism, specific information of deep portions can be acquired.

In addition, in order to effectively generate a photoacoustic wave, light must be irradiated in a sufficiently short period of time in accordance with thermal characteristics of the object. When the object is a living organism, a pulse width of the pulse light generated by the light source is preferably equal to or less than 200 nanoseconds and more preferably equal to or less than 100 nanoseconds.

Moreover, while light can be irradiated to deep portions of a living organism by using a solid state laser capable of producing output of several 10 millijoules or more per pulse as the light source, when measuring a vicinity of a surface of the living organism, a semiconductor laser, a light-emitting diode, and the like with relatively low irradiation energy may be used.

The light source unit 101 may be constituted by a single light source or a plurality of light sources. In addition, when the light source unit 101 is constituted by a plurality of light sources, the plurality of light sources may be constituted only by light sources emitting light with a same wavelength band or may be constituted so as to include light sources emitting light with different wavelength bands.

Furthermore, the light source may be a so-called wavelength-variable light source capable of varying a central wavelength. As a wavelength-variable light source, a laser capable of oscillating a wavelength in a near infrared range such as a titanium sapphire laser or an optical parametric oscillator (OPO) which utilizes optical parametric oscillation can be preferably used.

The luminous flux control unit 102 is a unit which controls an incidence position of a luminous flux on a fiber incident end surface.

Specifically, the luminous flux control unit 102 can be configured using a reflecting mirror (such as a galvanometer mirror) which changes an angle of a luminous flux or a transmitting optical component (such as a parallel plate) which causes light to be translated. In addition, an incidence position may be made successively changeable using simple moving means such as a stage. Alternatively, the luminous flux control unit 102 may be configured to control an incidence position of a luminous flux on a fiber incident end surface by changing a shape of the luminous flux. Examples of the luminous flux control unit 102 include a structure capable of changing an incidence position to a bundle fiber by changing a position where light emitted from a light source is shielded. Examples of such a shielding structure include a rotating body which rotates around an axis provided so as to be consistent with a travel direction of light. As the rotating body rotates around the axis, a shielded position on an optical path changes and, in turn, the incidence position changes.

In FIG. 1, arrows drawn by solid lines indicate a travel direction of a luminous flux.

A luminous flux generated at the light source unit 101 has a shape, a position, and the like thereof changed by the luminous flux control unit 102 and reaches an incident end of the bundle fiber 103.

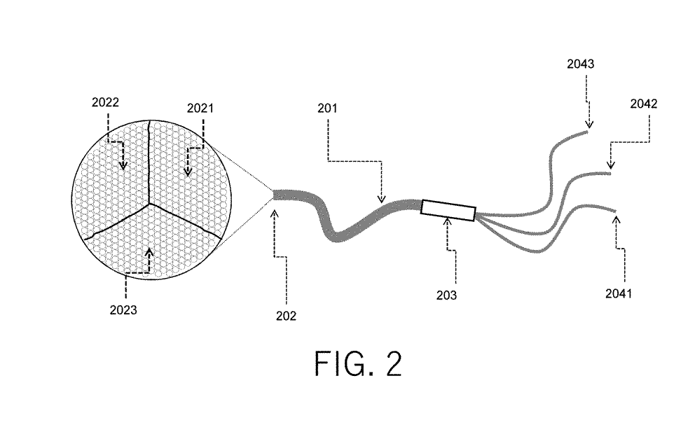

A relationship between a luminous flux and an incident end surface of a bundle fiber will now be described with reference to FIG. 2.

A bundle fiber 201 is constructed by bundling a plurality of fiber strands each with a diameter of 50 to 200 m. A bundle fiber is configured by bundling 100 to 10,000 fiber strands or several 100 to several 1000 fiber strands depending on an incident light size and a diameter of the fiber strands. A material of the fiber favorably conforms to a wavelength of the incident light source and has low transmission loss.

In the present embodiment, a unit obtained by bundling an assembly of fiber strands will be referred to as a strand group. A bundle fiber according to the present embodiment is configured so as to include a plurality of strand groups.

In addition, in the present embodiment, each strand group is arranged so that a plurality of regions are formed on the incident end surface of a bundle fiber. In the example shown in FIG. 2, a bundle fiber is constituted by three strand groups and the strand groups respectively construct regions represented by reference numerals 2021 to 2023. Moreover, the strand group corresponding to the region 2021 will be referred to as a strand group A, the strand group corresponding to the region 2022 will be referred to as a strand group B, and the strand group corresponding to the region 2023 will be referred to as a strand group C.

Each strand group is individually branched at the mixing branch portion 203, the strand group A is connected to an exit end 2041, the strand group B is connected to an exit end 2042, and the strand group C is connected to an exit end 2043.

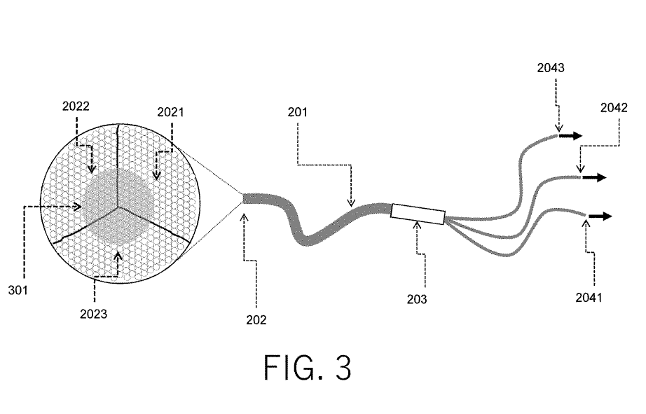

FIG. 3 is a diagram showing an example of a case where light is irradiated on an incident end surface of a bundle fiber. Reference numeral 301 denotes a position of light incident to an incident end 202 via a luminous flux control unit. As shown, when light is irradiated in a pattern uniformly straddling the respective strand groups A to C, light with approximately uniform intensity (approximately ⅓ of incident light) is respectively emitted from the exit ends 2041 to 2043.

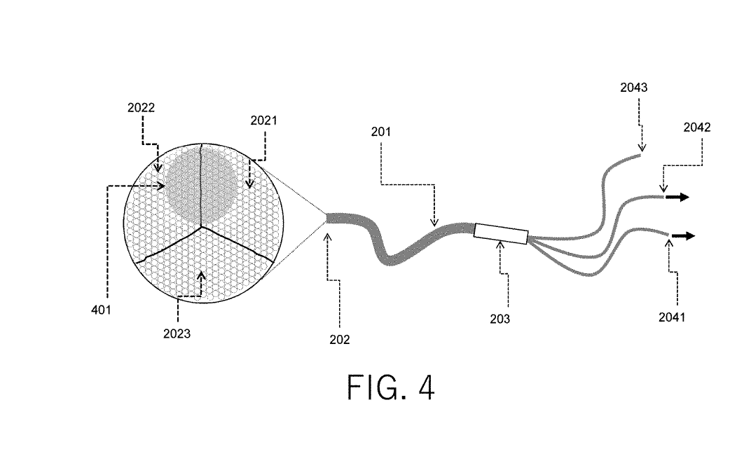

FIG. 4 is a diagram showing another example of a case where transmitted light is irradiated. Reference numeral 401 denotes a position of light incident to the incident end 202 via a luminous flux control unit. As shown, when light is irradiated in a pattern uniformly straddling the strand groups A and B, light with approximately uniform intensity (approximately ½ of incident light) is respectively emitted from the exit ends 2041 and 2042.

In addition, when light is caused to be incident to any one of the strand groups A to C (not shown), a pattern in which light is emitted from any of the exit ends 2041 to 2043 can be formed.

Moreover, in FIG. 2, an entirety of incident regions is given a circular shape and each incident region is given a shape created by equally dividing the circle. However, a configuration in which the incident end surface has a circular shape and incident regions are divided into fan shapes created by dividing the circle into three parts at a center thereof as shown is merely described as an example of the present invention. The number of divisions, the number of incident regions, a shape of the incident end surface, and the like are not limited thereto.

In addition, the irradiation patterns described above are merely examples and a position and intensity of light emitted from an exit end side can be controlled to various patterns by selecting (changing) an incidence position of a luminous flux on the incident end surface.

Furthermore, the number of divisions, the shape of the entirety of the exit regions, the number of exit regions, and the like on the exit region side can also be configured in various ways. As an example, in a similar manner to the incident regions, the entirety of the plurality of exit regions may be given a circular shape and each exit region can be given a shape created by dividing the circle into equal parts.

The plurality of fiber strands constituting each strand group are mixed in each strand group and arranged at random positions on an exit end surface. In other words, light incident to a certain fiber strand arranged in an incident region is emitted from a random position in a corresponding exit region. Specifically, on a cross-section on the exit end side of light of a bundle fiber, a plurality of exit regions respectively corresponding to the strand groups are arranged in a different layout from the plurality of incident regions. In addition, a configuration is adopted in which positions of the exit ends need not be changed even when the exit regions change in accordance with a change made to the incident regions as described above.

Accordingly, since strands to which light with high intensity is incident and strands to which light with low intensity is incident are mixed, a light distribution on each exit end surface becomes uniform even if a light distribution of a luminous flux on the incident end surface is not uniform.

As described above, with the light transmitting apparatus according to the first embodiment, a plurality of strand groups are arranged on a cross-section on the incident end side so as to form a plurality of incident regions, and an exit end corresponding to each strand group is provided. In addition, transmitted light is caused to be selectively incident to each of the plurality of incident regions. Accordingly, by slightly changing an incidence position of light on the incident end surface, an irradiation pattern of light irradiated to an object can be greatly changed.

In particular, unlike a periphery of an exit end, since a periphery of the luminous flux control unit 102 has a high degree of freedom in terms of installation space and is hardly subjected to spatial constraints, irradiation patterns with a high degree of freedom can be formed.

Moreover, while three strand groups are used in the present embodiment, more complex irradiation patterns can be formed by increasing the number of strand groups (in other words, the numbers of incident regions and exit ends). In addition, the incident end may be provided in plurality. Accordingly, an even wider variety of irradiation patterns can be formed.

Furthermore, when a bundle fiber includes a plurality of different incident ends, the luminous flux control unit 102 may be configured to divide light emitted from the light source by a number matching the number of incident ends and to respectively control incidence positions of the divided light. To this end, a branching optical element such as a polarizing beam splitter can also be used. In addition, in order to change a shape of light on the incident end surface, a circular luminous flux emitted from the light source may be changed into an elliptical shape using a cylindrical lens or the like.

Furthermore, a non-point-symmetric luminous flux may be rotated before being caused to be incident to the incident end surface of the bundle fiber.

### Second Embodiment

The second embodiment represents a photoacoustic apparatus including the light transmitting apparatus according to the first embodiment.

The photoacoustic apparatus according to the second embodiment is an apparatus utilizing a photoacoustic effect in which an acoustic wave generated inside an object by irradiating the object with light is received and characteristic information of the object is acquired as image data. Characteristic information refers to information related to a characteristic value corresponding to each of a plurality of positions inside the object which is generated using a received signal obtained by receiving a photoacoustic wave.

Characteristic information acquired by photoacoustic measurement is a value reflecting an absorption rate of optical energy. For example, characteristic information includes a generation source of acoustic waves generated by light irradiation, initial sound pressure inside an object, an optical energy absorption density or an optical energy absorption coefficient derived from initial sound pressure, or a concentration of substances constituting tissue. In addition, a distribution of oxygen saturation can be calculated by obtaining a concentration of oxygenated hemoglobin and a concentration of reduced hemoglobin as concentrations of substances. Furthermore, a glucose concentration, a collagen concentration, a melanin concentration, a volume fraction of fat or water, and the like are also obtained.

In addition to numerical value data, characteristic information may also be acquired as distribution data. In other words, distribution data on a distribution of light absorption coefficients, a distribution of oxygen saturation, and the like may be acquired. Furthermore, characteristic information may be acquired in an image data format.

Acoustic waves include waves called sonic waves, ultrasonic waves, and photoacoustic waves, and refer to elastic waves generated inside an object when the object is irradiated with light (electromagnetic waves) such as near infrared light. The photoacoustic apparatus according to the present embodiment is an apparatus which acquires object information regarding the inside of an object and which is mainly used for the purposes of diagnosing a malignant tumor, a vascular disease, and the like, performing a follow-up observation of chemotherapy, and the like of a human or an animal. Therefore, a living organism or, more specifically, a human or an animal is assumed as an object and a part such as a breast, a finger, or a limb of the human or the animal is assumed as a diagnostic object part.

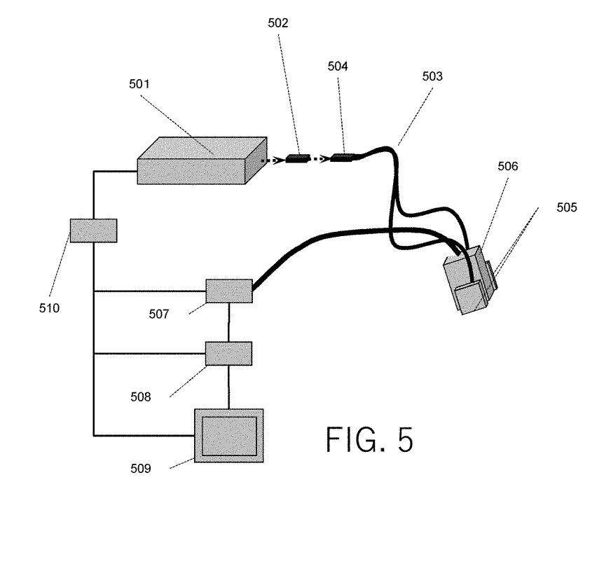

FIG. 5 is a configuration diagram of a photoacoustic apparatus according to the present embodiment.

The photoacoustic apparatus according to the present embodiment includes the light transmitting apparatus according to the first embodiment or, more specifically, a laser apparatus 501 as a light source unit, a luminous flux control unit 502, and a bundle fiber 503. The bundle fiber 503 includes an incident end 504 and an exit end 505.

In addition, the photoacoustic apparatus according to the present embodiment includes an ultrasonic detecting unit 506 which is an ultrasonic probe (an acoustic detector) and, by scanning the ultrasonic detecting unit 506, acquires a photoacoustic signal from a living organism that is the object. Furthermore, the photoacoustic apparatus according to the present embodiment includes a signal processing unit 507, a data processing unit 508, a display unit 509, and a control apparatus unit 510. Details of the respective apparatus units will be provided later.

Laser light oscillated by a light source is irradiated on a living organism via a light transmitting apparatus. In the present embodiment, each exit end of a bundle fiber is arranged in advance at a prescribed position.

Moreover, an irradiation position of light with respect to an object can be restricted to a certain degree by fixing a position of a fiber exit end. In contrast, the present embodiment enables a desired irradiation pattern to be selected by controlling an incidence position of light to an incident end with a luminous flux control unit.

In the present embodiment, the ultrasonic detecting unit 506 and the exit end 505 from which is emitted light to irradiate an object are arranged close to each other, enabling measurements to be performed while changing a positional relationship between the ultrasonic detecting unit and the light irradiation position. Accordingly, an optimal irradiation pattern can be selected in accordance with a state of a living organism that is a measurement target. Examples of irradiation patterns will be described in Examples 1 to 4 to be described later.

While the ultrasonic detecting unit 506 is constituted by a transducer using a piezoelectric phenomenon, a transducer using optical resonance, a transducer using a variation in capacity, or the like, the ultrasonic detecting unit 506 is not particularly limited to those described herein. As a transducer, any of a single element or a plurality of elements in an array may be used. In particular, when using a plurality of sensor elements, favorably, sensitivity, a position, an orientation, and the like of each element is appropriately set. A fiber exit end of the light transmitting apparatus may be directly mounted to an inner wall of a container shape, but mounting a fiber exit end via an optical member such as lens or a diffuser plate which forms a luminous flux shape in accordance with an object also represents a favorable embodiment.

In addition, in order to eliminate an effect of reflection or attenuation of acoustic waves, an acoustic matching material such as an acoustic matching gel, water, and oil may be provided between the object and an ultrasonic sensor to cause acoustic coupling. Furthermore, in order to detect acoustic signals from a wide range, the ultrasonic detecting unit 506 may be configured so as to be movable on a stage and to scan a surface of the object.

The signal processing unit 507 is a unit which amplifies an acquired electrical signal and converts the amplified electrical signal into a digital signal.

The signal processing unit 507 may be constituted by an amplifier which amplifies a received signal, an A/D converter which digitalizes an analog received signal, a memory such as a FIFO which stores a received signal, and an arithmetic circuit such as an FPGA chip. Alternatively, the signal processing unit 507 may be constituted by a plurality of processors or arithmetic circuits.

The data processing unit 508 is a unit which, based on a digitalized signal (hereinafter, a photoacoustic signal), acquires object information such as a light absorption coefficient and oxygen saturation of the inside of an object. Specifically, a three-dimensional initial sound pressure distribution inside the object is generated from a collected electrical signal. The initial sound pressure distribution can be generated using, for example, a universal back-projection (hereinafter, UBP) algorithm or a Delay and Sum algorithm.

In addition, the data processing unit 508 generates three-dimensional light distribution information of the inside of an object based on information regarding an amount of light irradiated on the object. Three-dimensional light distribution information can be acquired by solving a light diffusion equation from information related to two-dimensional light intensity distribution. Furthermore, a light absorption coefficient distribution inside the object which is object information can be obtained using the initial sound pressure distribution inside the object generated from a photoacoustic signal and three-dimensional light distribution information generated from a light intensity distribution of an irradiating unit. In addition, an oxygen saturation distribution inside the object can be obtained by computing a light absorption coefficient distribution at a plurality of wavelengths.

The data processing unit 508 can be constituted by a computer including a CPU, a RAM, a nonvolatile memory, and a control port. Each module is controlled as a program stored in the nonvolatile memory is executed by the CPU. The data processing unit may be a general-purpose computer or an exclusively designed work station. A multi-core CPU or the like can be used as the CPU.

The display unit 509 is a unit which displays information acquired by the data processing unit and processed information based on the acquired information, and is typically a display apparatus. The display unit 509 may include a plurality of display units and may be configured so as to be capable of parallel display.

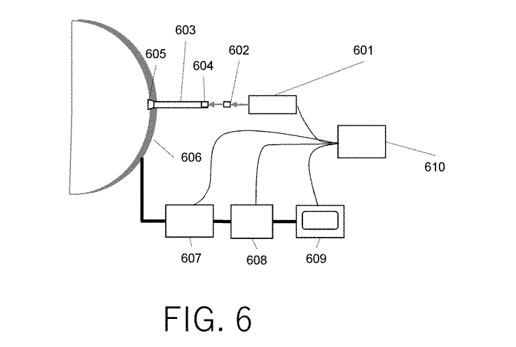

FIG. 6 is a modification of the photoacoustic apparatus according to the present embodiment. In the present modification, an ultrasonic detecting unit 606 is constituted by a plurality of ultrasonic sensors arranged on an inner wall of a container shape covering an object (typically, a breast). In addition to the ultrasonic detecting unit 606, the photoacoustic apparatus according to the present modification includes a laser apparatus 601, a luminous flux control unit 602, a bundle fiber 603 having an incident end 604 and an exit end 605, a signal processing unit 607, a data processing unit 608, a display unit 609, and a control apparatus unit 610. Since the respective units have been described earlier, detailed descriptions thereof will be omitted.

### Example 1

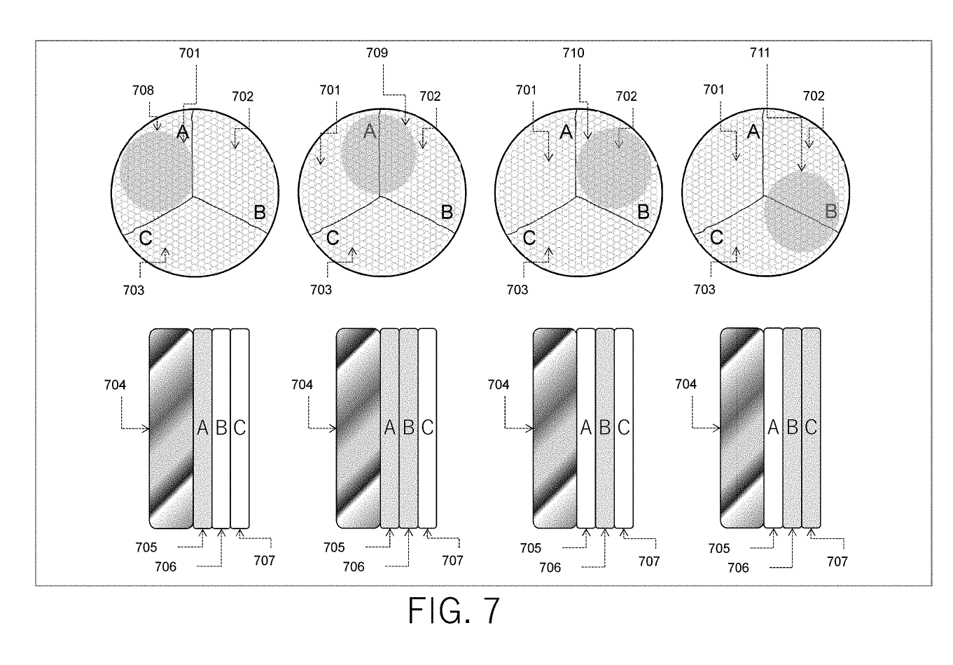

Next, a specific example of irradiating an object with light using the photoacoustic apparatus according to the second embodiment will now be described. FIG. 7 is a diagram showing a relationship between an incidence position of light on a fiber incident end surface and an exit position of light at a fiber exit end.

An upper half of FIG. 7 is a diagram representing cross-sections of a bundle fiber. In the present example, fiber strand groups are respectively grouped into a strand group A 701, a strand group B 702, and a strand group C 703 so that three incident regions are constructed on the fiber incident end surface.

A lower half of FIG. 7 is a diagram of the ultrasonic detecting unit 704 being observed from a direction in which the ultrasonic detecting unit is pressed against an object. In the present example, at the fiber exit end, an exit end surface 705 corresponding to the strand group A 701, an exit end surface 706 corresponding to the strand group B 702, and an exit end surface 707 corresponding to the strand group C 703 are respectively arranged so as to be stacked in a longitudinal direction of the ultrasonic detecting unit 704.

According to the present example, for example, by having the luminous flux control unit perform control so that a luminous flux emitted from the laser apparatus is only incident (reference numeral 708) to the strand group A 701, light can be irradiated from a region (the region 705) which is closest to the ultrasonic detecting unit 704.

In addition, by causing a luminous flux to be incident to a position (reference numeral 709) which straddles the strand group A 701 and the strand group B 702, light can be simultaneously irradiated from a wider region (the regions 705 and 706) than the region 705.

In a similar manner, by causing a luminous flux to be incident (reference numeral 710) only to the strand group B 702, light can be irradiated only from a region (the region 706) which is separated from the ultrasonic detecting unit 704.

In addition, by causing a luminous flux to be incident to a position (reference numeral 711) which straddles the strand group B 702 and the strand group C 703, light can be simultaneously irradiated from a wider region (the regions 706 and 707) than the region 706.

Moreover, in FIG. 7, a shape formed by an entirety of the plurality of incident regions is a circle and a shape formed by an entirety of the plurality of exit regions or, in other words, a shape of a region totalizing the exit end surfaces 705 to 707 is an approximate rectangle. However, a shape formed by the entirety of the plurality of exit regions is not limited to an approximate rectangle.

### Example 2

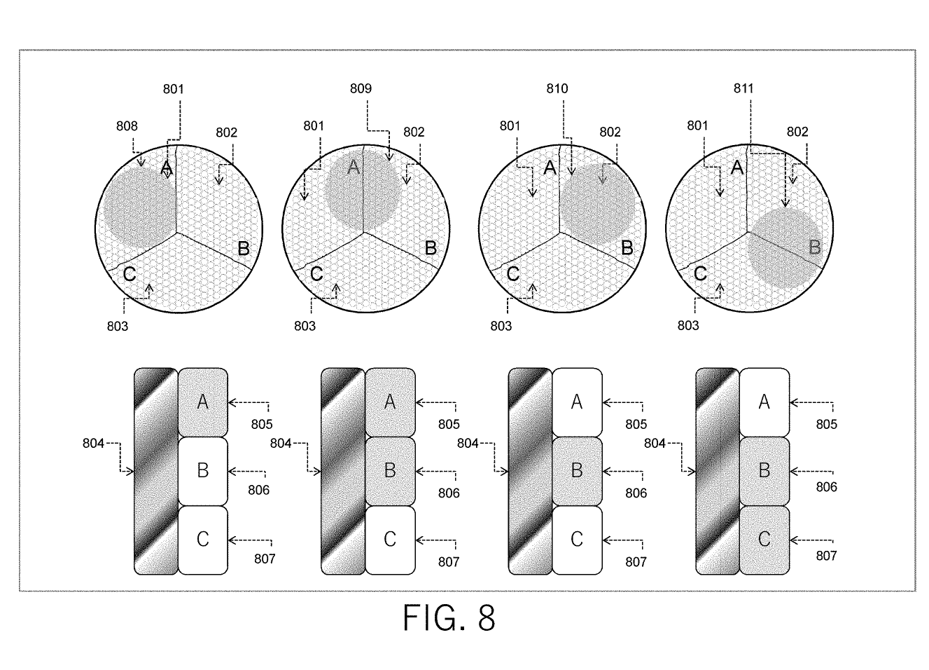

FIG. 8 is a diagram showing a relationship between an incidence position of light and an exit position of light according to Example 2.

While Example 2 is similar to Example 1 in that fiber strand groups are grouped so that three incident regions are constructed on a fiber incident end surface, Example 2 differs from Example 1 in an arrangement of exit end surfaces.

In the present example, at the fiber exit end, an exit end surface 805 corresponding to a strand group A 801, an exit end surface 806 corresponding to a strand group B 802, and an exit end surface 807 corresponding to a strand group C 803 are respectively arranged so as to be stacked along a longitudinal direction of an ultrasonic detecting unit 804.

According to the present example, for example, by having the luminous flux control unit perform control so that a luminous flux emitted from the laser apparatus is only incident (reference numeral 808) to the strand group A 801, light can be irradiated from a region (the region 805) which is arranged in an upper part on a plane of paper.

In addition, by causing a luminous flux to be incident to a position (reference numeral 809) which straddles the strand group A 801 and the strand group B 802, light can be simultaneously irradiated from a wider region (the regions 805 and 806) than the region 805.

In a similar manner, by causing a luminous flux to be incident (reference numeral 810) only to the strand group B 802, light can be irradiated only from an intermediately arranged region (the region 806).

In addition, by causing a luminous flux to be incident to a position (reference numeral 811) which straddles the strand group B 802 and the strand group C 803, light can be simultaneously irradiated from a wider region (the regions 806 and 807) than the region 806.

### Example 3

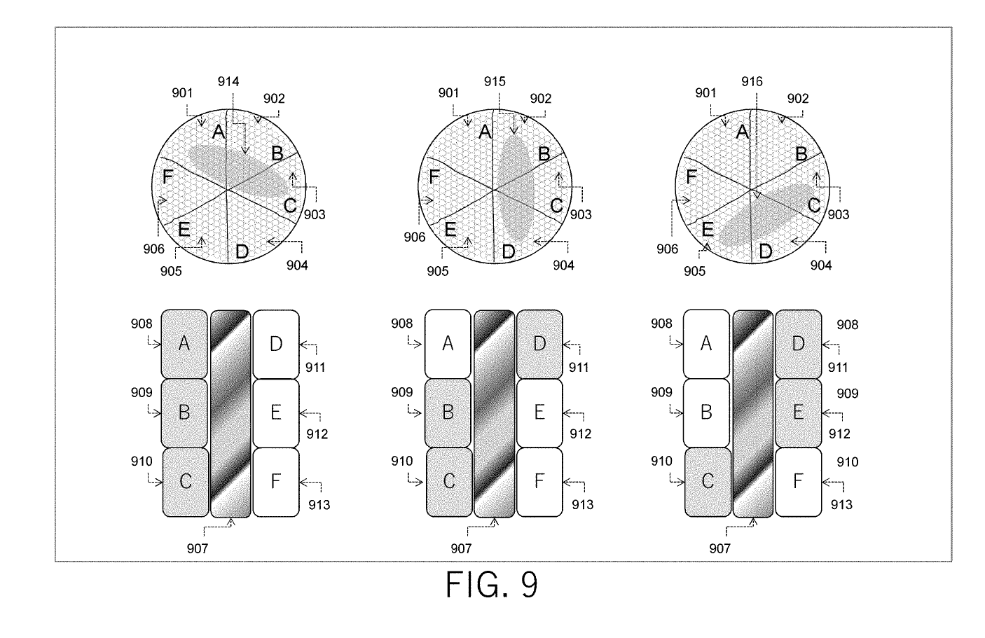

FIG. 9 is a diagram showing a relationship between an incidence position of light and an exit position of light according to Example 3.

In Example 3, fiber strand groups are grouped so that six incident regions are constructed on a fiber incident end surface. In addition, six exit regions are arranged on a fiber exit end surface.

In the present example, fiber strand groups are respectively grouped into a strand group A 901, a strand group B 902, a strand group C 903, a strand group D 904, a strand group E 905, and a strand group F 906 so that six incident regions are constructed on the fiber incident end surface.

In addition, at the fiber exit end, exit end surfaces 908 to 913 corresponding to the strand groups 901 to 906 are respectively arranged on both sides along a longitudinal direction of an ultrasonic detecting unit 907.

According to the present example, for example, by having the luminous flux control unit perform control so that a luminous flux emitted from the laser apparatus is incident (reference numeral 914) to the strand groups 901 to 903, light can be irradiated from a left side (reference numerals 908 to 910) of the ultrasonic detecting unit.

In addition, by causing a luminous flux to be incident to a position (reference numeral 915) which straddles the strand groups B 902 to D 904, light can be simultaneously irradiated from the regions 909, 910, and 911.

In a similar manner, by causing a luminous flux to be incident to a position (reference numeral 916) which straddles the strand groups C 903 to E 905, light can be simultaneously irradiated from the regions 910, 911, and 912.

As described above, according to Examples 1 to 3, by changing a shape of a luminous flux incident to an incident end surface of a bundle fiber (in other words, changing a position at which a luminous flux is incident on the incident end surface), an irradiation position of light with respect to an ultrasonic detecting unit can be controlled. Accordingly, for example, light can be irradiated while avoiding a region where a strong photoacoustic signal which may interfere with measurement (for example, a region where a mole or the like is present) is generated and accuracy of photoacoustic measurement is improved.

### Example 4

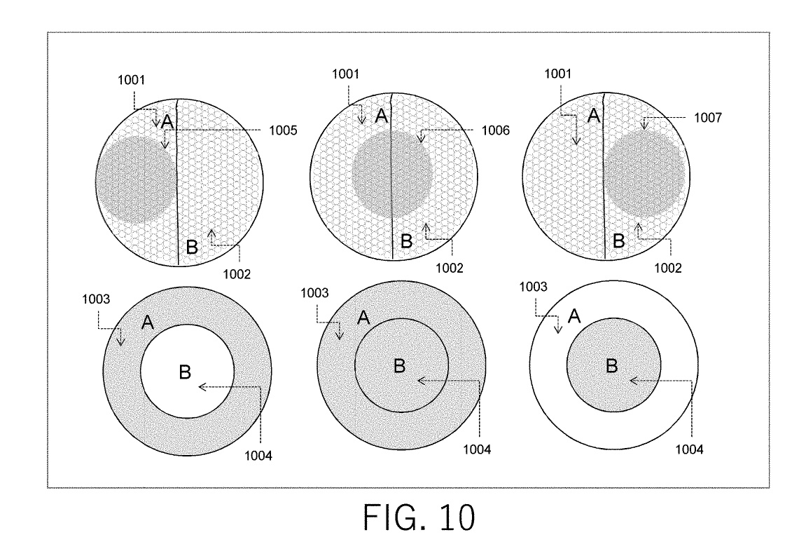

FIG. 10 is a diagram showing a relationship between an incidence position of light and an exit position of light according to Example 4.

An upper half of FIG. 10 is a diagram representing cross-sections of an incident end of a bundle fiber, and a lower half of FIG. 10 is a diagram representing cross-sections of an exit end of a bundle fiber.

In Example 4, fiber strand groups are respectively grouped into a strand group A 1001 and a strand group B 1002 so that two incident regions are constructed on the fiber incident end surface.

In addition, two exit regions are arranged on a fiber exit end surface. In the present example, at the fiber exit end, an exit end surface 1003 corresponding to the strand group A 1001 and an exit end surface 1004 corresponding to the strand group B 1002 are respectively concentrically arranged. In other words, incident regions and exit regions are arranged in different layouts. Specifically, a shape formed by an entirety of the plurality of incident regions is a circle, a shape formed by an entirety of the plurality of exit regions is also a circle, and the plurality of exit regions are concentrically arranged.

Even in Example 4, an irradiation position of light with respect to an object can be changed by controlling an incidence position of a luminous flux. For example, by having the luminous flux control unit perform control so that a luminous flux emitted from the laser apparatus is only incident (reference numeral 1005) to the strand group 1001, light can be irradiated from the annular region 1003. In addition, by having the luminous flux control unit perform control so that a luminous flux is only incident (reference numeral 1007) to the strand group 1002, light can be irradiated from the central region 1004. In a similar manner, light can be irradiated from all regions by causing a luminous flux to be incident to both strand groups 1001 and 1002.

Example 4 is particularly favorably performed by a photoacoustic apparatus in a mode such as that shown in FIG. 6. With a photoacoustic apparatus for performing cancer screening, a strong photoacoustic signal may be generated from pigment tissue in a periphery of a nipple which may interfere with measurement. In such a case, selecting a donut-shaped irradiation pattern when necessary enables irradiation of light to the nipple to be avoided and, accordingly, enables accuracy of photoacoustic measurement to be improved.

### Other Embodiments

It is to be understood that the descriptions of the respective embodiments merely present examples of the present invention and, as such, the present invention can be implemented by appropriately modifying or combining the embodiments without departing from the spirit and the scope of the invention.

For example, the present invention can be implemented as a light transmitting apparatus which includes at least a part of the components described above. In addition, the present invention can also be implemented as an object information acquiring apparatus which includes at least a part of the components described above. The processes and units described above can be implemented in any combination thereof insofar as technical contradictions do not occur.

Embodiment(s) of the present invention can also be realized by a computer of a system or apparatus that reads out and executes computer executable instructions (e.g., one or more programs) recorded on a storage medium (which may also be referred to more fully as a non-transitory computer-readable storage medium) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc DVD), or Blu-ray Disc BD)™), a flash memory device, a memory card, and the like.

In addition, while a positional relationship between a plurality of exit ends and an ultrasonic detecting unit is fixed in the description of the embodiments, configuring exit ends to be movable using simple moving means enables an even wider variety of irradiation patterns to be formed.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-26677, filed on Feb. 16, 2017, which is hereby incorporated by reference herein in its entirety.

What is claimed is:

1. A light transmitting apparatus, comprising:
a bundle fiber configured to include a plurality of strand groups, each of which is an assembly of fiber strands; and
a luminous flux control unit which controls an incidence position of light incident on an incident end-side cross-section of the bundle fiber, wherein
in the bundle fiber,
the plurality of strand groups are arranged so as to respectively form a plurality of incident regions on the incident end-side cross-section, and a plurality of exit regions respectively corresponding to the strand groups are arranged in a different layout from the plurality of incident regions on a cross-section on an exit end side of light of the bundle fiber, and
the luminous flux control unit is configured to be capable of changing a region, to which transmitted light is to be incident, among the plurality of incident regions by controlling the incidence position of light.

2. The light transmitting apparatus according to claim 1, wherein
the plurality of exit regions are arranged at mutually separated positions.

3. The light transmitting apparatus according to claim 1, wherein
on the exit end-side cross-section of the bundle fiber, the plurality of strand groups are arranged so as to respectively form a plurality of exit regions.

4. The light transmitting apparatus according to claim 1, wherein
the luminous flux control unit is capable of simultaneously irradiating light to two or more of the incident regions.

5. The light transmitting apparatus according to claim 1, wherein
light incident to a fiber strand arranged in the incident region is emitted from a random position in the exit region corresponding to the incident region.

6. The light transmitting apparatus according to claim 1, wherein
the bundle fiber is configured such that, in accordance with a change of the incident region, the exit region changes without changing a position of the exit end.

7. The light transmitting apparatus according to claim 1, wherein
the luminous flux control unit controls the incidence position by changing a position at which light emitted from a light source is shielded.

8. The light transmitting apparatus according to claim 1, wherein
a shape formed by an entirety of the plurality of incident regions is a circle, and each of the incident regions has a shape created by equally dividing the circle.

9. The light transmitting apparatus according to claim 1, wherein
a shape formed by an entirety of the plurality of exit regions is a circle, and each of the exit regions has a shape created by equally dividing the circle.

10. The light transmitting apparatus according to claim 1, wherein
a shape formed by an entirety of the plurality of incident regions is a circle, and a shape formed by an entirety of the plurality of exit regions is an approximate rectangle.

11. The light transmitting apparatus according to claim 1, wherein
a shape formed by an entirety of the plurality of incident regions is a circle, a shape formed by an entirety of the plurality of exit regions is a circle, and the plurality of exit regions are concentrically arranged.

12. An object information acquiring apparatus, comprising:
the light transmitting apparatus according to claim 1; and
an acoustic wave detector which detects acoustic waves generated when light emitted from the light transmitting apparatus irradiates an object.

13. The object information acquiring apparatus according to claim 12, wherein
the plurality of exit regions are arranged at respectively different positions with respect to the acoustic wave detector.

14. A light transmitting apparatus including a bundle fiber configured to include a plurality of strand groups, each of which is an assembly of fiber strands, wherein
in the bundle fiber,
the plurality of strand groups are arranged so as to respectively form a plurality of incident regions on an incident end-side cross-section, a plurality of exit regions respectively corresponding to the strand groups are arranged in a different layout from the plurality of incident regions on an exit end side, and
in accordance with a change of the incident region, the exit region changes without changing a position of the exit end.

15. A method of transmitting light using a bundle fiber in which a plurality of fiber strand groups are arranged so as to respectively form a plurality of incident regions on an incident end-side cross-section and a plurality of exit regions respectively corresponding to the fiber strand groups are arranged in a different layout from the plurality of incident regions on an exit end side,
the method comprising:
selecting the incident region to which transmitted light is to be incident; and
performing light transmission by causing light to be incident to the selected region, wherein
in accordance with the selected incident region, the light is emitted from the exit end side in a different light distribution without changing a position of the exit end.
